(19) United States
(12) Patent Application Publication
(10) Pub. No.: US 2023/0127171 A1
(43) Pub. Date: Apr. 27, 2023

(54) DISSOLVABE WET WIPE

(71) Applicant: MCCORMACK INNOVATION LIMITED, Glenrothes (GB)

(72) Inventor: BRIAN JAMES MCCORMACK, By Kircaldy Fife (GB)

(21) Appl. No.: 18/083,154

(22) Filed: Dec. 16, 2022

Related U.S. Application Data
(63) Continuation-in-part of application No. 17/252,565, filed on Dec. 15, 2020.

(30) Foreign Application Priority Data
Jun. 15, 2018 (DE) 202018002808.8

(51) Int. Cl. A61K 8/02; A61K 8/34

(52) U.S. Cl. CPC A61K 8/0208; A61K 8/34

(57) ABSTRACT

A moist or wet wipe comprising or consisting of a non-woven wet laid material which contains or consists of soluble material and is coated or impregnated with cleaning or moisturising substances.

### CROSS-REFERENCE TO RELATED APPLICATIONS

This application is a continuation-in-part of Application Ser. No. 17252565, filed 15 Dec. 2020, which in turn is the National Stage of International Application No. PCT/GB2018/053218, filed Jun. 11 2018.

### BACKGROUND

The present invention relates to a dissolvable wet wipe.

Wet wipes are widely used sanitary items. Wet wipes are single use cleaning clothes which come in sealed packs and are disposed of after use. Wet wipes comprise a woven/non-woven textile material which holds or is coated with a cleaning fluid or agent. Presently wet wipes are manufactured using fibrous sheets made of materials such as Polyester, Polypropylene of Rayon fibres. None of these materials are environmentally friendly. They can take up to 50 years to break up and are a threat to marine life. Wet wipes are responsible for 93% of blocked UK sewage pipes. The waste from them is also widely distributed in the environment, for instance 5,463 wet wipes were recovered from 116 m² of the River Thames in April 2018. There are currently legislative initiatives to ban wet wipes totally.

The present invention seeks to address this problem.

### SUMMARY

Accordingly a first aspect of the present invention is directed to a moist or wet wipe comprising or consisting of a woven or non-woven material which contains or consists of soluble material and is coated or impregnated with cleaning substances.

Advantageously the wet wipe consists entirely of non-woven or woven material which is dissolvable.

This provides the advantage that the wet wipe is dissolvable will not block sewerage systems and meets the Sewage Network Abuse Protocol criteria and is not harmful to the environment in general.

### BRIEF DESCRIPTION OF THE DRAWINGS

Examples of wet wipes made in accordance with the present invention will now be described hereinbelow with reference to the attached drawing, in which:

FIG. 1 is a plan view of a wet wipe according to the present invention;

FIG. 2 is a plan view of a further wet wipe according to the present invention;

FIG. 3 is a plan view of a further wet wipe according to the present invention; and

FIG. 4 is a photograph of wet wipes according to the first and second aspect of the present invention.

### DETAILED DESCRIPTION

Preferably the base material of the wipe is a non woven, wet laid, synthetic yarn, advantageously of the Polyvinyl Alcohol (PVA) material. This provide the advantage that the wipe is easily dissolvable by cold, warm or hot water with no aid of any chemical agents. In a preferred embodiment the wipe designed to be strong enough to be functional for the purposes intended.

The PVA has a number of forms but generally has the formula (—CH₂—CH(OH)— n)(CH(OH)-n)(—CH₂-CH(OCOCH₃)-m).

Physical properties are:

Weight; 25-40 gsm

Thickness; 75-150 microns

Tensile Strength; 140-750 N/m

Elongation; 5-10%

Tear Resistance; 2-3 N

Cold Water Dispersibility (initial); 3-5 secs

Cold Water Dispersibility (final) 3-5 minutes

In a preferred embodiment the wet wipe has a rhomboid, rectangular or square shape. The wet wipe is suitable to be dispensed from a sealed pack.

Advantageously the soluble material is comprised of webs of water soluble polyvinyl alcohol fibres and a water soluble resin fixably adhered to the fibres of which the fabric is composed to form a chemical lace. This has the advantage that these fibres are readily available and will dissolve in water in a reasonable time.

In a preferred embodiment the material of the wet wipe includes a number of separate pieces of standard (non dissolvable) wet wipe material, such as woven or non-woven synthetic fibres, which are stitched together or joined together by intermingling with soluble thread using needling or joined together with soluble adhesive to create a patchwork.

The pieces in this patchwork arrangement can be of any suitable rhomboid shape but preferably the pieces are squares. Advantageously they have a size of 1 to 2 inches. This provides the advantage that they fragment into small segments when exposed to water. If flushed down a sewage system there is little risk of “roping type” effects which leads to blockages.

In a preferred embodiment the wipes are impregnated with a stabilising liquid such as almond oil, mineral oil, baby oil or such like. This maintains the integrity soluble fibres until exposed to water.

Advantageously dependent upon its use, the material finish and texture can be made to produce a soft finish or a mildly abrasive finish for example with baby oil for the removal of cradle cap.

Preferably the surface of the wipe can be changed by compression during manufacturing to produce grip points for more effective abrasive for example the removal of dead skin.

In a preferred embodiment the wipe has a medical application especially for the removal of adhesive residue from the skin for example in the case of stoma care.

Advantageously when saturated with 100% alcohol the wipe can be used for electrical equipment cleaning and the maintenance of clean rooms.

Preferably the wipes can be made in any dimension but for medical wipes this is normally 5″×5″. For personal care, 7″×8″ and larger for industrial applications,

Advantageously the wipes can be manufactured with material (soluble or non-soluble) that is perforated, for example in 2 to 4 inch segments, to weaken the structure and create breakaway points.

In a preferred embodiment for ease of use the wipe can be made in a glove form, especially for application of essential oils in beauty therapy, massage and/or physiotherapy.

Preferably when packed, sealed and tested, the wipe stability is adapted to meet industry standard shelf life criteria.

An advantageous list of additives that can be use and are compatible with the wipe material include:
a) wipe for adhesive residue removal from skin; C9-C12 Iso Paraffin greater than 90%, Triticum Vulgare, Lavandula Angustifolia, Melaleuca Alternifolia, Aloe Barbadensis (Aloe) Leaf Extract, Citric Acid, Sodium Benzoate, Potassium Sorbate, Linalool, Limonene.
b) Wipe for Electrical Equipment and maintenance of Clean Rooms; 100% alcohol
c) Personal care wipes
   i) Coconut oil wipe; 100% coconut oil
   ii) Almond oil wipe; 100% almond oil
   iii) Baby wipe; mineral oil and fragrance.

Preferably approximately 3 ml of each additive is required for each wipe.

In a preferred embodiment the wipes are free from boric acid.

A second aspect of the present invention is directed to a moist or wet wipe consisting of 100% single laid one directional polyvinyl alcohol (PVA) fibres with high directional strength coated or impregnated with cleaning, beauty, or therapeutic substances.

Advantageously the wipe contains reduced levels of fibrous product with a GSM of around 25 GSM. This has the advantage that the resultant wet wipe gives a soft feel to the human skin.

The wipe has the advantage that it has high machine directional strength to make easy any manufacturing going forward for wipes and reducing the chance of breaking in the manufacturing process.

Preferably the wet wipe is subject to napping during production to soften the material. In the Napping process to soften the material, fibres are pulled to the surface using steel wires of constant length, density and aggressiveness.

Another method of softening the material during manufacturing is to introduce Calendaring rollers. These rollers with the one direction laid fibrous polyvinyl alcohol (PVA) soften the material due to their high pressure and different types of surface giving a desired softness.

The target area of use for this very soft wipe is varied as it can be impregnated with various natural oils and chemicals.

If a wipe according to this aspect is put down a toilet it will dissolve within one minute or if it is put in landfill will degrade within 7 days.

There are a wide number of uses for this soft dissolving wipe such as the application of cosmetics as the wipe has the ability to retain and distribute lotions.

A softer wet wipe as described above could be used in the medical field to apply lotions to broken skin or remove medical adhesive for instance from stoma flange areas which often have broken skin.

Wipes according to the second aspect of the present invention can be used as:

Medical Wipes
Medical
   Stoma Care—Adhesive Remover Wipes
   Hemorrhoid wipes
   Anti Virus and Bacteria Infection wipes
   Pre Injection swabs
   Medical Equipment wipes

General Wipes
Industrial
   All trades, surface cleaning wipes
   Electrical equipment wipes
Personal care
   Facial/Body wipes.
   Tanning wipes
   Mosquito Repellent wipes
   CBD oil wipes
   Toilet wipes
Household
   Surface Cleaning wipes
   Plant care wipes
Commercial
Outdoors
   BBQ/Tinder lighters
Veterinary
   Tack wipes

A wet wipe consisting of or comprised entirely of non-woven or woven dissolvable fibres is not illustrated but would comprise a rhomboid, rectangular or square shape as shown in FIG 1. This provides for a wet wipe which dissolved completely in a short period on exposure to water.

FIG. 1 shows a plan view of a wet wipe 10. The wet wipe 10 is composed of multiple squares 12 of synthetic woven or nonwoven fabric. This fabric can be of a standard material used in currently available wet wipes. These squares 12 form a chequered pattern. The edges of the squares 12 are joined to the adjacent square or squares 12 by stitching with soluble fibre or thread. Such that horizontal rows 14 of stitching stretch across the wet wipe 10 with substantially perpendicular vertical rows 16 of stitching which are spaced apart such that they are essentially at the edges of the squares 12. Alternatively the joins 14,16 could be achieved by soluble adhesive as opposed to stitching.

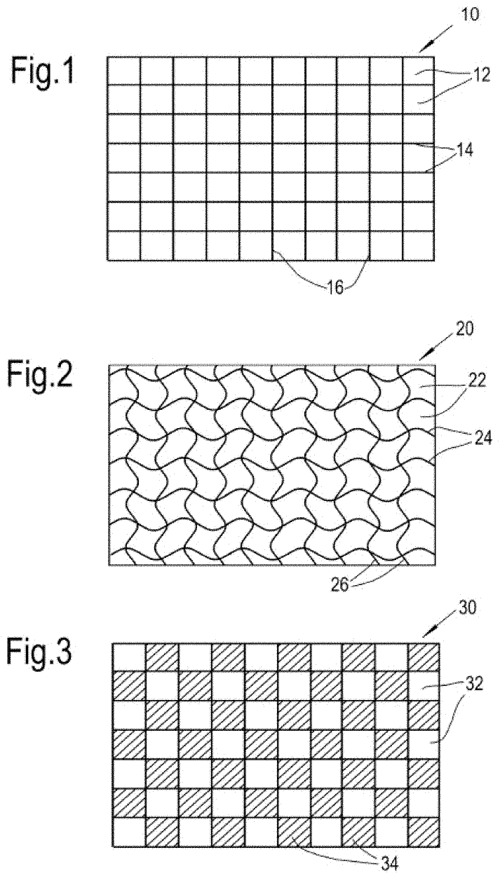

FIG. 2 shows a plan view of a similar wet wipe 20. The wet wipe 20 is composed of nonwoven fabric divided into squares 22. This fabric can be of a standard material used in currently available wet wipes. The squares 22 to form a chequered pattern. The edges of the squares 22 are joined to the adjacent squares 22 by sections of non-woven soluble fibres which are pressed into the squares 22 by needling. Such that horizontal rows 24 of non-woven soluble fibres stretch across the wet wipe 20 with substantially perpendicular vertical rows 26 of nonwoven soluble fibres.

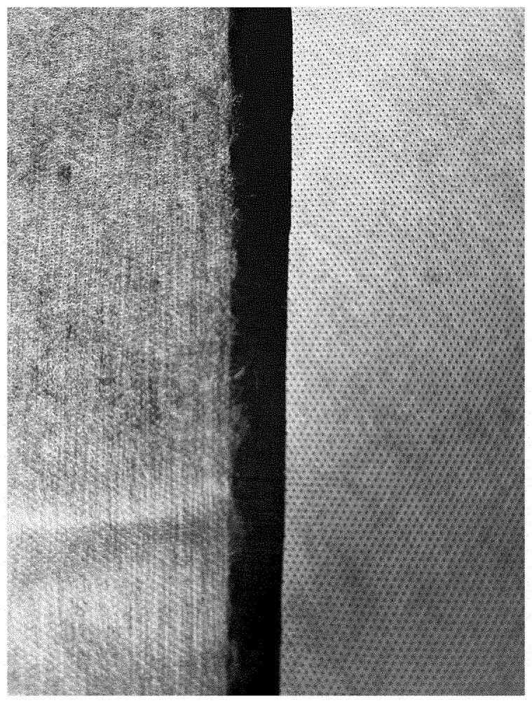

FIG. 3 shows a plan view of a wet wipe 30 comprising a chequered pattern of squares 32 and 34. The squares 32 comprise synthetic non-woven fabric and squares 34 comprise non-woven soluble fibres which are joined with the squares 32 during the needling process creating the fabric of the wipe 30. The non-soluble fibres can be of a standard material used in currently available wet wipes.

The wet wipes are coated or impregnated with almond oil or mineral oil in order to introduce integrity to the soluble threads/fibres or adhesive such that they do not disintegrate until placed in water.

The moist or wet wipe can obviously be used to convey various materials for a cleaning purposes, such as anti-septic materials, soap, cleaning materials, alcohol and such like in order to meet desire cleaning the purpose.

An example of a wet wipe made in accordance with the second aspect of the present invention is made laying webs of soluble Polyvinyl Alcohol Fibres onto a mesh conveyor and impregnated with water soluble resins. The material then goes through a series of rollers to bond the resins to the Polyvinyl Alcohol Fibres. The material is then dried. After drying the resulting material is soluble. The material can then be cut to the appropriate size for the wipes.

FIG. 4 shows a wet wipe manufactured according to the second aspect of the present invention on the left and the first aspect on the right. The wipe on the right according to the first aspect has a smooth surface with a regular pattern of holes between the PVA fibres. This pattern of holes improves the carrying of cleaning, beauty, or therapeutic substances such as lotion etc. The wipe on the left has a furrier aspect with a surface of fibres brought out by the softening treatments giving a safter feel. This fibres also hold cleaning, beauty, or therapeutic substances such as lotion etc.

Uses of such a wipe include:
General purpose wipes including baby wipes. and swish cloth wipes to fit on cleaning mop pads;
Medical wipes for the application of chemicals to kill bacteria and viruses;
Pre-injection wipes impregnated with Alcohol which lowers the skins Bacteriological count prior to injection;
Cosmetics wipes for the application and removal of all cosmetics; and
Material can also be used in the production of Hydration sheet face masks.

1. A moist or wet wipe comprising or consisting of a woven or non-woven material which contains or consists of soluble material and is coated or impregnated with cleaning substances.

2. A moist or wet wipe according to claim 1, in which the wet wipe consists entirely of non-woven or woven material which is dissolvable.

3. A moist or wet wipe according to claim I , in which the wipe is made of non-woven, wet laid, synthetic yarn.

4. A moist or wet wipe according to claim 1, in which of the material is a Polyvinyl Alcohol (PVA) material.

5. A moist or wet wipe according to claim 1, in which the wipe designed to be strong enough to be functional for the purposes intended.

6. A wet wipe according to claim 1, in which the wet wipe has a rhomboid, rectangular or square shape.

7. A moist or wet wipe according to claim 1. in which the wet wipe is suitable to be dispensed from a sealed pack.

8. A moist or wet wipe according to claim 1, in which the soluble material is comprised of webs of water soluble polyvinyl alcohol fibres and a water soluble resin fixably adhered to the fibres of which the fabric is composed to form a chemical lace.

9. A moist or wet wipe according to claim 1, in which the material of the wet wipe includes a number of separate pieces of standard (non dissolvable) wet wipe material, such as woven or non-woven synthetic fibres, which are stitched together or joined together by intermingling with soluble thread using needling or joined together with soluble adhesive to create a patchwork.

10. A moist or wet wipe according to claim 9, in which the pieces in this patchwork arrangement can be of any suitable rhomboid shape

11. A moist or wet wipe according to claim 10, in which the pieces are squares, advantageously they have a size of 1 to 2 inches.

12. A moist or wet wipe according to claim 1, in which the wipes are impregnated with a stabilising liquid such as almond oil, mineral oil, baby oil or such like.

13. A moist or wet wipe according to claim 1, in which the material finish and texture can be made to produce a soft finish or a mildly abrasive finish.

14. A moist or wet wipe according to claim 1, in which the surface of the wipe is compressed during manufacturing to produce grip points for more effective abrasive.

15. A moist or wet wipe according to claim 1, in which the wipe is for a medical application, including the removal of adhesive residue.

16. A moist or wet wipe according to claim 1, in which when saturated with alcohol is used for electrical equipment cleaning and the maintenance of clean rooms.

17. A moist or wet wipe according to claim 1, in which the dimension of the wipe is 5″×5″ for medical applications and 7″×8″ for personal and industrial applications.

18. A moist or wet wipe according to claim 1, in which the wipe is in a glove form.

19. A moist or wet wipe according to claim 1, in which the wipe stability is adapted to meet industry standard shelf life criteria.

20. A moist or wet wipe according to claim 1, in which the wipe material includes one or more of the following additives depending on use;
a) wipe for adhesive residue removal from skin; C9-C12 Iso Paraffin greater than 90%, Triticum Vulgare, Lavandula Angustifolia, Melaleuca Alternifolia, Aloe Barbadensis (Aloe) Leaf Extract, Citric Acid, Sodium Benzoate, Potassium Sorbate, Linalool, Limonene.
b) Wipe for Electrical Equipment and maintenance of Clean Rooms; 100% alcohol
c) Personal care wipes
i) Coconut oil wipe; 100% coconut oil
ii) Almond oil wipe; 100% almond oil
iii) Baby wipe; mineral oil and fragrance.

21. A moist or wet wipe according to claim 20, in which approximately 3 ml of each additive is required for each wipe.

22. A moist or wet wipe consisting of 100% single laid one directional polyvinyl alcohol (PVA) fibres with high directional strength coated or impregnated with cleaning, beauty, or therapeutic substances.

23. A moist or wet wipe according to claim 22, containing reduced levels of fibrous product with a GSM of around 25 GSM.

24. A moist or wet wipe according to claim 22, in which the wipe has high machine directional strength to make easy any manufacturing going forward for wipes and reducing the chance of breaking in the manufacturing process.

25. A moist or wet wipe according to claim 22, in which the wipe is subject to napping during production to soften the material.

26. A moist or wet wipe according to claim 22, in which the material of the wipe is softened during manufacturing by using Calendaring rollers.

27. A moist or wet wipe according to claim 22, in which the wipe is impregnated with various natural oils and chemicals.
